(19) United States
(12) Patent Application Publication
(10) Pub. No.: US 2020/0315539 A1
(43) Pub. Date: Oct. 8, 2020

(54) RAPID EXCHANGE CATHETER SYSTEM FOR FRACTIONAL FLOW RESERVE MEASUREMENT

(71) Applicant: MURATA MANUFACTURING CO., LTD., Nagaokakyo-shi, Kyoto (JP)

(72) Inventor: Heikki KUISMA, Helsinki (FI)

(21) Appl. No.: 16/652,833

(22) PCT Filed: Nov. 5, 2018
(86) PCT No.: PCT/IB2018/001223

§ 371 Date: Apr. 1, 2020

(30) Foreign Application Priority Data
Nov. 8, 2017 (FI) 20175997

(51) Int. Cl. A61B 5/00; A61B 5/0215; A61B 5/026

(52) U.S. Cl. CPC A61B 5/6852; A61M 2025/0002; A61B 5/026; A61B 5/02158

(57) ABSTRACT

The present invention relates to an elongated rapid exchange catheter configured to measure fractional flow reserve of a patient. The catheter comprises a shaft and a distal portion coupled to a distal end of the shaft. The distal portion comprises a lumen configured for inserting a guide wire, and an entry via in a side wall of the lumen for inserting the guide wire into the lumen. The entry via is disposed closer to the proximal end of the distal portion than the distal end of the distal portion. The catheter further comprises plurality of sensing devices comprising at least one distal sensing device disposed at the distal portion and a proximal sensing device. The at least one distal sensing device and the proximal sensing device are disposed at a predefined distance from each other along the longitudinal dimension of the catheter. The proximal sensing device is disposed on the proximal side of the entry via.

### FIELD

The present invention relates to an apparatus related to measuring blood flow in a blood vessel. More particularly, the present invention relates to a catheter for measuring fractional flow reserve for determining a degree of stenosis in a cardiac artery. This type of catheters is also known in the art as cardiovascular catheters.

### BACKGROUND

Fractional flow reserve measurement is an established method for determining the extent of a stenosis in cardiac arteries.

In a prior art procedure for determining a stenosis illustrated in the FIG. 1, a guide wire 110 is inserted to a major artery for example at the groin or at the wrist with an uncalibrated distal pressure sensor 100 at the tip of the guide wire 110. The guide wire 110 is pushed into the aorta 50 and a fluid filled catheter 120 is inserted on the guide wire 110 so that it extends to the aorta 50. A calibrated external pressure sensor 130 obtains the pressure values at the distal end of the catheter via the fluid filling the catheter 120. The pulsating blood pressure in the aorta 50 is facilitated to calibrate the distal pressure sensor 100 disposed on the guide wire 110. After calibration in the aorta 50, the patient may be given medication that will cause maximum hyperaemia. The catheter 120 and the guide wire 110 with the calibrated distal pressure sensor 100 are pushed into the proximal side 55 of the cardiac artery under investigation and the pressure readings are recorded. Before the guide wire with the distal pressure sensor 100 is pushed through a stenosis 51 in the cardiac artery so that the pressure sensor 100 becomes disposed on the distal side 56 of the stenosis 51. This second position of the distal pressure sensor 100 is marked with the reference 100′. The pulsating pressure in the proximal side 55 of the stenosis is recorded with the external pressure sensor 130 at the same time to recording the pulsating pressure on the distal side 56 of the stenosis at the tip of the guide wire with the distal pressure sensor 100. The average values of the two pulsating pressure readings may then be calculated, and a ratio of the distal pressure reading at the tip of the sensor and the proximal pressure reading is calculated. This ratio is called the Fractional Flow Reserve or FFR and is used to determine the degree of the stenosis and thus the need for any treatment. The whole operation is done under an X-ray imager so that the position of the guide wire and/or the catheter can be seen at all times. The catheter may be made visible in an X-ray image by assembling a radiopaque zone at the distal end of the catheter.

FIG. 2 illustrates simultaneous pressure readings received from two different pressure sensors. The pressure reading curve 200 represents the measured pressure on the proximal side 55 of a stenosis, while the pressure curve 210 represents the measured pressure values on the distal side 56 of the stenosis 51. An average or a median of the two detected pressure values may be used to calculate the FFR.

Guide wires are typically tiny and equipped with a flexible tip which facilitates navigation in blood vessels to reach a lesion or vessel segment. Once the tip arrives at its destination, it acts as a guide, which a larger and typically stiffer catheter can rapidly follow for easier delivery to the measurement and/or treatment site. A guide wire may also serve as a visual guide to the operating personnel, as it is typically made of material which is visible in X-ray imager. Thus, the position of the guide wire can be seen at all times.

While a catheter is typically inserted with aid of X-rays, it is also important to minimize the time of exposure of the patient to the radiation, which is known to be harmful. Therefore, it is beneficial to use a so called rapid exchange catheter, in which a guide wire only travels through a lumen in a distal portion of the catheter. A rapid exchange catheter enables shortening the radiation exposure up to 30% compared to a normal catheter with a full-length guide wire.

Currently, the FFR measurement solution disclosed in the FIG. 1 is not is not available using a rapid exchange catheter because the rapid exchange catheters don't have a lumen in the proximal portion of the catheter, also known as the shaft.

### DESCRIPTION OF THE RELATED ART

U.S. Pat. No. 4,815,472 discloses a sensor device for determining the pressure difference over the stenosis on a catheter with two sensors having a predetermined distance along the catheter. The sensor device comprises no provision for a guide wire. The lumen of the catheter doesn't extend to the tip and is only used for electrical wiring from the sensors to the external instrumentation.

U.S. Pat. Nos. 4,762,129, 5,040,548 and 5,738,667 disclose rapid exchange catheters, in which only a relatively short segment of the distal end of the catheter is advanced over the guide wire.

International patent application publication WO2014/181274 discloses an example of an elongated pressure sensor structure that may be applied to a sensor used with the present invention.

### SUMMARY

An object is to provide an apparatus so as to solve the problem of combining both accurate fractional flow reserve measurement and agile blood vessel navigation. The objects of the present invention are achieved with an apparatus according to the characterizing portion of claim 1.

The preferred embodiments of the invention are disclosed in the dependent claims.

The present invention is based on the idea of disposing a plurality of sensing devices into a catheter and positioning the sensing devices both for measuring changes in pressure over a lesion and for facilitating good agility and mechanical robustness of the catheter.

The present invention has the advantage that the distance between distal and proximal sensing devices may be increased with the placement of the proximal sensing device, and also risk of harming the proximal sensing device or its wiring with the guide wire is reduced by the disposal of the proximal sensing device. A benefit from a greater distance between the distal and proximal sensing devices is that the proximal device is always far away from the lesion so that the proximal pressure reading is not affected the lesion in case of multiple or complex lesions.

According to a first aspect, an elongated rapid exchange catheter is provided, that is configured to measure fractional flow reserve of a patient. The catheter comprises a shaft and a distal portion coupled to a distal end of the shaft. The distal portion comprises a lumen configured for inserting a guide wire, and an entry via in a side wall of the lumen for inserting the guide wire into the lumen. The entry via is disposed closer to the proximal end of the distal portion than the distal end of the distal portion. The catheter further comprises plurality of sensing devices comprising at least one distal sensing device disposed at the distal portion and a proximal sensing device. The at least one distal sensing device and the proximal sensing device are disposed at a predefined distance from each other along the longitudinal dimension of the catheter. The proximal sensing device is disposed on the proximal side of the entry via.

According to a second aspect, the proximal sensing device is disposed at the distal portion of the catheter between the entry via and the proximal end of the distal portion.

According to a third aspect, the proximal sensing device is disposed in a recess within the volume of the shaft at or near the distal end of the shaft.

According to a fourth aspect, the proximal sensing device is disposed in a recess at a junction coupling the shaft and the distal portion.

According to a fifth aspect, any sensing device disposed at the distal portion is disposed in a recess within the volume of the side wall of the distal portion.

According to a sixth aspect, the sensing devices comprise a pressure sensor and an interface circuitry that performs an analog-to-digital conversion to at least one signal received from the pressure sensor.

According to a seventh aspect, the rapid exchange catheter comprises at least two distal sensing devices disposed at mutually different distances from the proximal sensing device.

According to an eighth aspect, one of the proximal and distal sensing devices comprise a flow sensor.

### BRIEF DESCRIPTION OF THE DRAWINGS

In the following the invention will be described in greater detail, in connection with preferred embodiments, with reference to the attached drawings, in which

FIG. 1 is an illustration of a FFR measurement apparatus as known in the art.

FIG. 2 illustrates outcome of a FFR measurement.

FIG. 3 illustrates a first example of a catheter.

FIG. 4 illustrates a second example of a catheter.

FIGS. 5a to 5d illustrate lateral, longitudinal views of a portion of the catheter with a lumen and electrical wiring disposed at the side wall.

FIG. 6 illustrates a longitudinal cross-section of a catheter.

FIG. 7 illustrates a rapid exchange catheter.

FIG. 8 illustrates a first embodiment of a rapid exchange catheter.

FIG. 9 illustrates a second embodiment of a rapid exchange catheter.

FIG. 10 illustrates a third embodiment of a rapid exchange catheter.

FIG. 11 illustrates a fourth embodiment of a rapid exchange catheter.

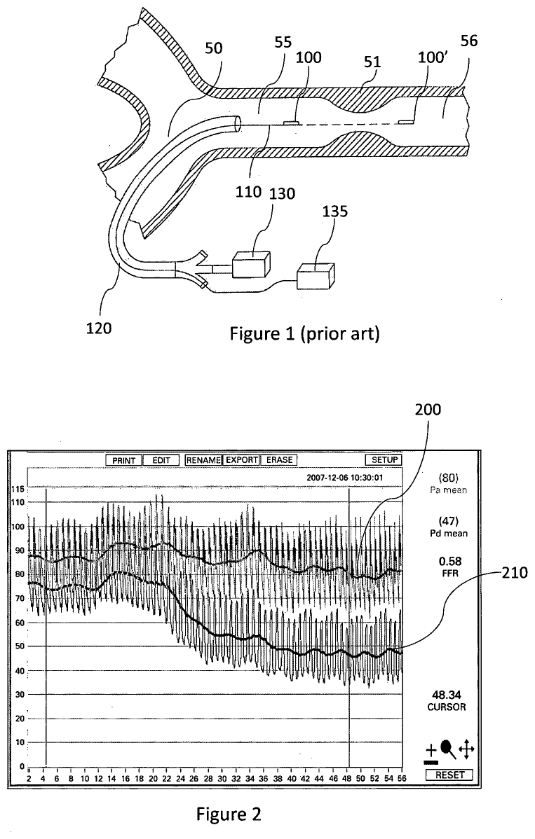

### DETAILED DESCRIPTION

As used in the term distal refers to parts of a catheter closest to the heart of a subject and also direction towards the heart of the subject. The term proximal refers parts of the catheter farthest from the heart of the subject that is examined with a catheter, as well as direction away from the heart. Thus, a user, for example a medical professional, may be in contact of the proximal end of the catheter while the distal end of the catheter refers to the end of the catheter that resides inside the patient when the catheter is in use. Likewise, a distal part of an artery is towards the heart when compared to a proximal part of the artery, and the proximal part of the artery is away from the heart when compared to the distal part.

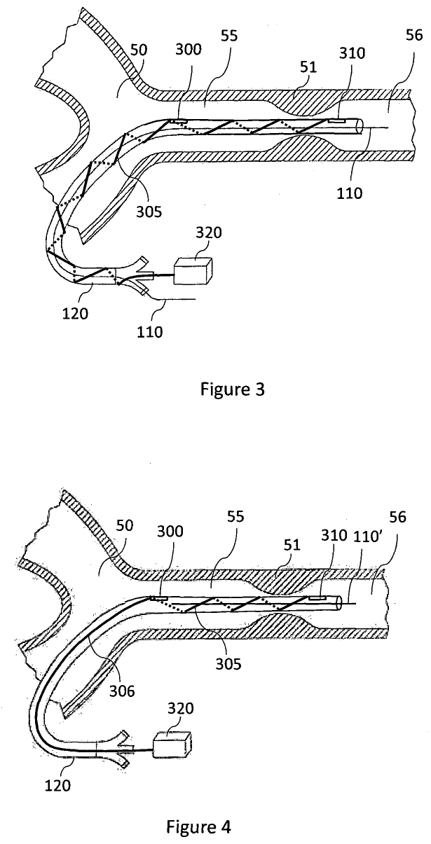

The FIG. 3 shows a first example of a catheter. The tubular catheter 120 is provided with a hollow inner space, also known as a lumen, for inserting a guide wire 110. The lumen inside may extend over the entire length of the catheter 120. A catheter 120 for FFR measurement may have a total length of approximately 180 cm. The longitudinal lumen of the tubular catheter 120 is defined by the side walls of the catheter 120. The transverse cross-section of the lumen is preferably circular to facilitate insertion of a circular guide wire 110.

The catheter 120 is equipped with at least two pressure sensing devices: a proximal sensing device 300 and a distal sensing device 310. The distal sensing device 310 is disposed at or near the distal end of the catheter 120 and the proximal sensing device 300 is disposed at a predetermined distance from the distal sensing device 310 along the longitudinal dimension of the catheter towards the proximal end of the catheter 120. The distance between the distal sensing device 310 and a proximal sensing device 300 may be rather large, for example between 10 and 30 cm, preferably between 15 and 25 cm, so that the proximal sensing device will always remain on the proximal side of complex or multiple lesions, preferably in or close to the aorta. In some embodiments, the catheter 120 may have more than one distal sensing devices 310, which may be disposed at different distances from each other to facilitate profiling of a complex lesion. In this case the distal sensing devices should be located with a distance from 1 cm to 5 cm from each other. In some embodiments, the one or more distal sensing devices 310 and the proximal sensing device 300 are pressure sensors. In some embodiments at least two of the plurality of sensing devices are pressure sensors and at least one of the sensing devices is a flow sensor. A flow sensor may be disposed at any portion of the catheter 120, since blood flow remains essentially constant on both sides of the stenosis. Combining readings of a flow sensor with pressure readings received from the at least two pressure sensors may further improve quantification of the stenosis, and in some cases, avoid necessity to use medication that will cause hyperaemia for purpose of the measurements. The sensing devices 300, 310 are preferably disposed in one or more recesses within the volume of the side wall of the catheter. The one or more recesses preferably open towards the outer periphery of the catheter 120 so that the pressure sensors may detect the pressure in the periphery of the catheter 120.

The guide wire 110 and the catheter 120 are pushed to a cardiac artery under investigation. The guide wire 110 may be first pushed all the way to the distal part 56 of the cardiac artery, past a stenosis 51 to be investigated, and the catheter 120 may then be inserted on the guide wire 110 so that the distal portion of the catheter 120 extends all the way to the distal part 56 of the cardiac artery. The distal sensing device 310 disposed at or near the distal end of the catheter 120 is configured to be inserted in the distal part 56 of the cardiac artery and the proximal sensing device 300 is configured to be inserted in the proximal part 55 of the cardiac artery so that the two sensing devices (300, 310) become disposed on opposite sides of a stenosis 51. The sensing devices (300, 310) are now readily positioned to measure the pressure readings on the two opposite sides of the stenosis along the cardiac artery, and the readings of the sensing devices (300, 310) may be used to determine the Fractional Flow Reserve FFR value.

Each of the sensing devices 300, 310 preferably comprises a pressure sensor and an interface circuitry disposed at the immediate vicinity of the respective pressure sensor. The pressure sensor and the respective interface circuitry are electrically connected to each other. The interface circuitry may receive an analog signal from the pressure sensor that represents or corresponds to a pressure detected by the pressure sensor. The analog signal may comprise for example a voltage, a current, a resistance or a capacitance. The interface circuitry preferably detects the analog signal and converts it into a digital signal that is carried to the proximal end of the catheter 120, out from the blood vessels, via an electrical wiring 305. The interface circuitry may be implemented as an application specific integrated circuit (ASIC). In order to facilitate insertion of the guide wire 110 through the lumen of the catheter 120, the electrical wiring 305 is preferably disposed at the side wall of the catheter 120 at least in the distal portion of the catheter. Insertion of the guide wire is facilitated by annular form of the cross section of the catheter at least in the distal portion of the catheter 120. The electrical wiring 305 may be disposed at the side wall essentially over the entire length of the catheter 120. By the term at the side wall we refer to disposal of the electrical wiring 305 on or in the side wall. The electrical wiring 305 may be disposed on the outer surface of the side wall. Electrical wiring 305 disposed on the outer surface of the side wall may comprise insulated wires, so that the electrical wiring 305 does not come into contact with bodily fluids when the catheter is in use. A non-limiting example of such insulated wires are so called magnet wires, also known as enamel wires, in which the conductor wire is coated with a very thin layer of insulation.

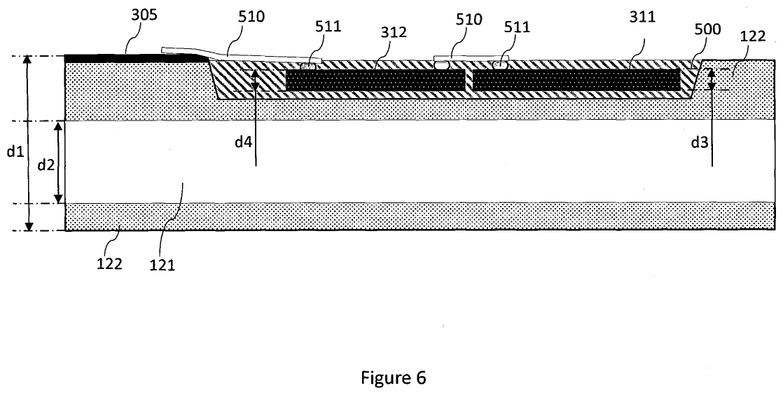

Alternatively, or in addition, the electrical wiring 305 may be embedded in the side wall, in other words into the volume of the side wall, thus protected from any contact with the bodily fluids. The electrical wiring 305 may comprise a plurality of electrical wires electrically isolated from each other. For example, there may be separate electrical wires for providing electrical power for operating the sensing device 300, for carrying one or more digital signals received from the sensing device 300, for carrying one or more digital signals for controlling the interface circuitry and/or for carrying signals between different parts of the sensing device 300, which parts will be discussed in more detail in relation to FIG. 6. The plurality of electrical wires of the electrical wiring 305 may be spread over the perimeter of the outer side wall or arranged parallel to each other. Preferably, pressure information provided by the plurality of sensing devices 300, 310 is carried by the electrical wiring 305 combined into a single digital signal.

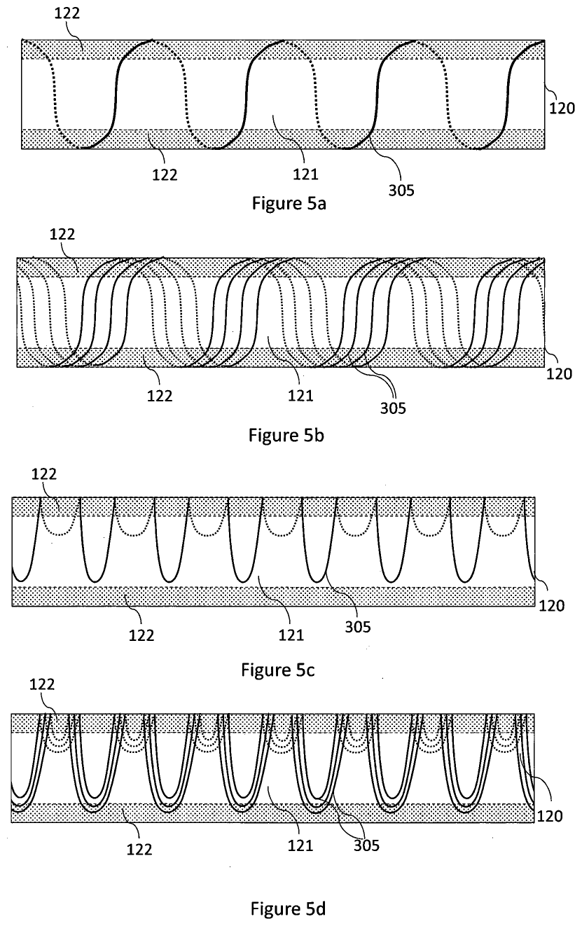

In order to facilitate flexibility of the catheter 120 in all directions, the electrical wiring 305 disposed at the side walls may be spiraled, as illustrated in the FIGS. 3, 4, 5a and 5b, or meandering, as illustrated in the FIGS. 5c and 5d. The spiraled electrical wiring 305 refers to wiring which travels diagonally along the side wall forming a spring-like spiral, encircling the lumen inside the catheter.

The electrical wiring 305 preferably carries at least one digital signal. The at least one digital signal may carry signals with information from all sensing devices 300, 310. The digital signal may comprise combined information from more than one sensing device 300, 310. The electrical wiring 305 preferably couples the at least one digital signal provided by the sensing devices 300, 310 towards a digital interface means 320 disposed at the proximal end of the catheter, which digital interface means 320 communicates the obtained sensor reading information carried by the signals towards external equipment, such as a computer, a monitor or a patient monitor. The digital interface means 320 preferably provides wireless communication towards the external equipment, but also a wireline connection may be applied. Wireless communication may be preferred, since it reduces amount of wiring required in the operation room. The digital interface 320 may be implemented using any suitable wireless communication technology known in the art. Preferably, a low-energy wireless communication is applied, such as standardized Bluetooth, Zigbee and UWB technologies, or one of a variety of proprietary radio frequency communication technologies. In hospital environment, especially in an operation room, minimizing the amount of cabling needed is preferable, since a great amount of cabling may impede movements of the personnel, cause disorder and confusion.

The FIG. 4 shows an embodiment of a catheter according to the invention. This embodiment represents a type of catheter 120 known in the art as a rapid exchange catheter, in which a lumen for a guide wire 110′ is provided only at the distal portion of the catheter 120. The guide wire 110′ of a rapid exchange catheter thus only travels within the distal portion of the catheter, and an entry via through a side wall of the lumen is arranged for the guide wire 110′ at the proximal part of the distal portion of the catheter 120. The distal portion is configured to facilitate entering the guide wire into the lumen at the entry via and to facilitate traveling of the guide wire within the lumen towards the distal end of the distal portion, which also forms the distal end of the catheter. The entry via may also be called entrance point. From the entry via towards the proximal end of the catheter, the guide wire 110′ remains outside the catheter (not shown). The rapid exchange catheter has typically a 25-30 cm long flexible distal portion with lumen and a stiff proximal portion with length around 150 cm without a lumen. The proximal portion may be called shaft. The proximal sensing device may be advantageously located close to the entrance point of the guide wire. Preferably the distance of the proximal sensing device to the entry via of the guide wire is less than 5 cm. The proximal sensing device may be located either on the distal portion of the catheter with the guide wire or on the proximal portion without a guide wire. Preferably the proximal sensing device is located on the flexible portion of catheter 120. The distance between the distal sensing device 310 and a proximal sensing device 300 may be rather large, for example between 10 and 30 cm, preferably between 15 and 25 cm, so that the proximal sensing device will always remain on the proximal side of the lesion or lesions, also in case of complex or multiple lesions. When in use, the proximal sensing device is preferably disposed in or close to the aorta.

The proximal portion of the catheter may also have a lumen, but since the proximal portion is not used for insertion of a guide wire, it may be utilized for example for inserting electrical wiring 306. In the distal portion of the catheter 120, the electrical wiring 305 is disposed at the side walls of the catheter 120 in the similar manner as explained in relation to the first embodiment, and the electrical wiring 305 at the distal portion may be spiraled or meandering. This way the lumen at the proximal portion is left free for insertion of the guide wire 110′. Both the electrical wiring 305 at the distal portion of the catheter 120 and the electrical wiring 306 in the lumen of the proximal portion of the catheter 120 may comprise a plurality of wires. The spiraled or meandering electrical wiring 305 may extend essentially over the entire length of the catheter as illustrated in the FIG. 3, or only over a portion of the length of the catheter as illustrated in the FIG. 4.

FIGS. 5a to 5d illustrate lateral, longitudinal side views of exemplary portions of the catheter 120 with a lumen 121. FIG. 5a illustrates electrical wiring 305 with a single, spiraling wire, whereas FIG. 5b illustrates electrical wiring 305 with three spiraling wires. The plurality of spiraling wires may be grouped parallel as in the FIG. 5b, or they may be spread over the periphery of the catheter 120 so that the distance between any two adjacent wires is maximized.

The meandering form of electrical wiring 305 indicates that the electrical wiring 305 bends back and forth at the side wall, but does not form a spiral circulating around the lumen inside the tubular catheter 120. The meandering electrical wiring 305 may form for example a sine-wave type pattern at the side wall as illustrated in the FIGS. 5c and 5d; or a zig-zag or sawtooth pattern. The FIG. 5c illustrates electrical wiring 305 with a single meandering wire, whereas the FIG. 5d illustrates electrical wiring 305 with three meandering wires disposed in parallel with each other. Alternatively, meandering wires may be spread over the periphery of the catheter 120 so that the distance between any two parallel wires may be maximized. The examples shown in the FIGS. 5a to 5d are illustrative only, not limiting the scope. Electrical wiring 305 may be implemented with any number of wires.

The FIG. 6 illustrates a longitudinal cross-section of a tubular portion of the catheter 120 at the location of the sensing device 300 or 310, showing the lumen 121 defined by the side wall 122. The sensing device 300, 310 comprises a pressure sensor 311 and an interface circuitry 312 disposed within the volume of a side wall 122 of the catheter 120.

The catheter 120 preferably comprises at least one tubular portion with an annular cross-section, in which a side wall defines an essentially circular circumferential perimeter and an essentially circular lumen. The annular cross-section may be concentric or eccentric. Preferably, at least the distal portion of the catheter with the sensing devices 310, 311 is tubular, with the lumen configured for inserting a guide wire. The outer diameter d1 of the essentially circular perimeter of the catheter 120 may be for example 0.66 mm, and the diameter d2 of the essentially circular lumen may be 0.33 mm. At the location of the sensing device shown in the FIG. 6, the diameter of the entire catheter construction may bulge slightly so that the outer diameter d1 may be up to approximately 1 mm. The lumen may be eccentric compared to the outer perimeter of the catheter 120. Eccentric disposal of the lumen 121 allows a thicker side wall 122 in a sector of the catheter, thus facilitating assembly of the sensing device 300, 310 as illustrated in the FIG. 6.

The distal, tubular part of a rapid exchange catheter may be 20-30 cm long. The pressure sensor 311 and the interface circuitry 312 of the sensing device 300, 310 shall be thin enough to allow assembly within the side wall 122 of the catheter 120 even in the thin and flexible distal portion of the catheter. In a typical catheter 120, thickness d3 of the pressure sensor 311 and thickness d4 of the interface circuitry 312 may be limited to a maximum value of 0.1 mm. For the same reason, the width of pressure sensor 311 and the interface circuitry 312 may be limited to a maximum value of 0.35 mm.

While the catheter 120 is a narrow but long device allowing, it allows installing elongate devices within its side wall 122. Thus, the length of the pressure sensor 311 and of the interface circuitry 312 may be several millimeters. Separate interface circuitry 312 and pressure sensor 311, and a mechanically flexible electrical interconnection 510 between the pressure sensor 311 and the interface circuitry 312 both facilitate flexibility of the catheter 120. While any chip area of the pressure sensor 311 and the interface circuitry 312 may not be increased too much by increasing the width of the chip, the length dimension may be utilized for increasing chip area needed to perform the intended functions. However, keeping the pressure sensor 311 and the interface circuitry 312 as separate chips enables a flexible interconnection between the two, which facilitates flexibility of the entire sensing device assembly. The pressure sensor may for example be implemented using a microelectromechanical sensor structure disclosed in the international patent application WO2014/181274.

The pressure sensor 311 and the interface circuitry 312 may be disposed in a recess disposed at the outer surface of the side wall 122. Preferably, the catheter 120 comprises a plurality of recesses so that each sensing device may be disposed in a different recess. Within the recess, there may be a protective coating 500 of for example silicone gel over or around the pressure sensor 311 and the interface circuitry 312. A layer of silicone gel may separate the pressure sensor and the interface circuit from the influence of blood, which is electrically conductive and would severely disturb the operation if it was allowed to enter into contact with the pressure sensor or the interface circuit. On the other hand, silicone gel may transmit the pressure of the blood undisturbedly to the sensor.

Silicone gel will absorb water from the blood and this absorbed water will change the dielectric coefficient of the gel and in the case of a capacitive sensor the capacitance of the sensor may be influenced by this change. It is, however possible to compensate this change by additional reference measurements of the interconnection capacitances. In some embodiments, the electrical contacts towards the pressure sensor 311 and the interface circuitry 312 are exposed towards the outer perimeter of the side wall 122 and thus towards the electrical interconnections 510 disposed at the periphery of the catheter 120. The outer surface of the protective coating 500 is preferably essentially aligned with the outer surface of the side wall 122. In some embodiments, only the electrical contacts of the interface circuitry 312 are exposed at the outer surface of the catheter 120, in other words, towards the periphery of the catheter 120, while the electrical connections via the interconnection bumps 511 and even the electrical interconnection 510 between the pressure sensor 311 and the interface circuitry 312 are disposed entirely inside the protective coating 500.

The pressure sensor 311 and the interface circuitry 312 configured to handle signals from the pressure sensor 311 are preferably disposed in the immediate vicinity of each other so that the distance for carrying small, sensitive analog signals from the pressure sensor 311 to the interface circuitry 312 is as short as possible. The preferably oblong or elongated pressure sensor 311 and the preferably oblong or elongated interface circuitry 312 may be disposed in-line along the longitudinal dimension of the catheter. Alternatively, the pressure sensor 311 and the interface circuitry 312 may be disposed radially side by side, disposed in one or more suitably formed installation recesses within the catheter side wall volume. The FIG. 6 illustrates the in-line disposal. A digital signal is less sensitive to interference than an analog signal received from the pressure sensor, and therefore, converting the small analog signal into a digital signal as close to the pressure sensor 311 as possible is preferred. Further, a plurality of digital signals may be multiplexed for transmission over the wiring. This way, less wires are needed for transmitting multiple pressure measurement results, and reduced number of wires may further facilitate agility of the catheter.

Contact pads of the pressure sensor 311 and the interface circuitry 312 may be electrically coupled with each other by any applicable interconnecting means 510, such as bond wires, a metal lead frame or via wiring disposed on a flexible circuit board. Likewise, the interface circuitry 312 may be electrically coupled to the electrical wiring 305 by any applicable interconnecting means 510, such as bond wires, metal lead-frame or wiring of a flexible circuit board. In the example of FIG. 6, the pressure sensor 311 and the interface circuitry 312 have a flip chip structure known in the art of semiconductor devices. Chip pads, in other words connection pads of electrical signals in or out from a flip chip semiconductor may be directly connected to external circuitry. Application of the flip chip technology allows beneficially exclusion of often bulky packages of the chips in the tiny space available for the measuring device in the recess within the catheter side wall 122. For coupling the chip pads to external circuitry, a flip chip device comprises interconnection bumps 511, which may comprise for example any of solder balls, gold stud bumps and copper pillars, disposed directly onto chip pads of the semiconductor die as known in the art. The interconnection bumps 511 are configured to be galvanically coupled to the interconnecting means 510. The interconnecting means 510 may be disposed to the periphery of the side wall 122. For example, the interconnecting means 510 may disposed on the outer surface of the side wall 122, outer surface of the protective coating 500 and/or on the electrical wiring 305, depending on the placement of the interconnecting means 510.

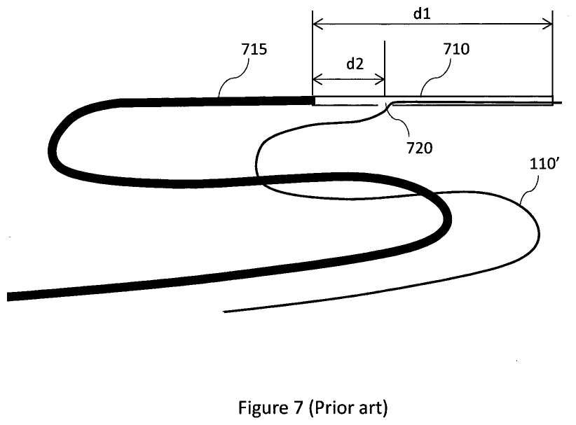

FIG. 7 illustrates a rapid exchange catheter as known in the art. The catheter comprises a flexible distal portion 710 with a lumen for a guide wire 110′ and a more rigid shaft 715, which may have a lumen, but not for the guide wire 110′. The shaft may have a lumen for example for electrical wiring. Length and thickness of the distal portion 710 is defined by physical characteristics of a heart and cardiac arteries, depending on the intended use and for example size of the patient. A typical rapid exchange catheter has a distal portion 710 with length d1 of 20-30 cm, while length of the shaft 715 may be about 1.5 m. The entrance via 720 in which the guide wire 110′ is configured to enter the lumen is typically closer to the proximal end than the distal end of the distal portion 710. For example, the entrance via 720 may be located at distance d2 from the proximal end of the distal portion 710, which distance d2 may be less than 10 cm, for example 8 cm.

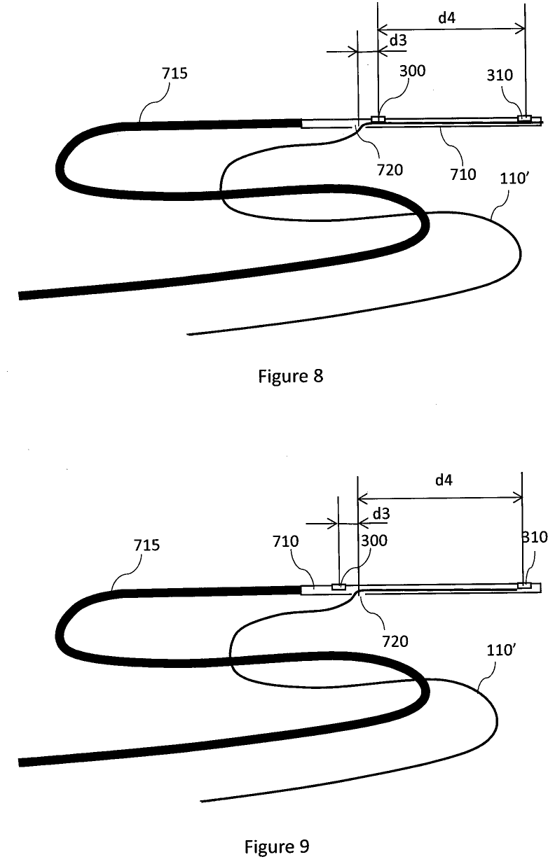

FIGS. 8 and 9 illustrate a first and a second embodiment of a rapid exchange catheter with a distal sensing device 310 and a proximal sensing device 300. In these embodiments, the proximal sensing device 310 is located close to the entry via for the guide wire 110′, the distance d3 between the entry via and the proximal sensing device is preferably less than 5 cm. In the first embodiment illustrated in the FIG. 8, the proximal sensing device 300 is disposed on the distal side or the entrance via 720, while in the second embodiment, the proximal sensing device 300 is disposed on the proximal side of the entrance via 720. The distance d4 between the distal sensing device 310 and the proximal sensing device 300 may be rather large, for example between 10 and 30 cm, preferably between 15 and 25 cm, so that the proximal sensing device will always remain on the proximal side of the lesion or lesions. The configuration of the FIG. 9 may be preferred, since while the proximal sensing device 300 and its wiring is on the proximal part of the distal portion 710 and on the proximal side of the entry via 720, risk of harming the proximal sensing device 300 or its wiring with the guide wire 110′ is reduced.

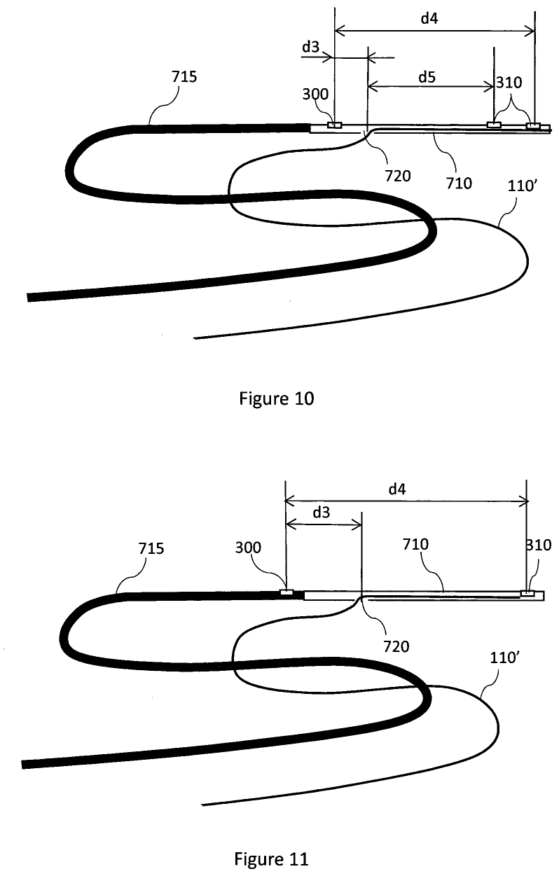

FIG. 10 illustrates a third embodiment of a rapid exchange catheter. In this embodiment, there are more than one distal sensing devices 310, in this example two. A first distal sensing device 310 is disposed at or near the distal end of the catheter, and it has a distance of d4 from the proximal sensing device 300. A second distal sensing device 310 is disposed further away from the distal end of the catheter, and it has a distance d5 from the proximal sensing device 300. Even more than two distal sensing devices may be disposed at the distal portion 710 at different distances from the distal end and the proximal sensing device 300. By having more than one distal sensing devices 310, it is possible to measure more than one pressure differences between different sensing devices. In the exemplary embodiment, it is possible to measure the pressure differences between either of the distal sensing devices 310 and the proximal sensing device 300 but also between the two distal sensing devices 310. Thus, it is possible to get more information from the measurements for example in case of more than one lesions or a long, non-uniform lesion.

FIG. 11 illustrates a fourth embodiment of a rapid exchange catheter. In this embodiment, the proximal sensing device 300 is disposed at the shaft part of the rapid exchange catheter. This way, the distance d4 between the at least one distal sensing device 310 and the proximal sensing device 300 may be further increased. The proximal sensing device 300 may be disposed within a recess formed in the volume of the shaft 715. In a further embodiment, the proximal sensing device 300 may be disposed in a recess formed at the junction between the distal portion 710 and the shaft 715. For example, there may be a coupling structure between the distal portion 710 and the shaft 715, and the proximal sensing device 300 may be disposed in a recess formed at the coupling structure.

Above illustrated embodiments are not restricting, and any combination of individual features of the embodiments may be implemented. For example, the plurality of distal sensing devices 310 illustrated in the FIG. 10 may be combined with any of the other embodiments.

It is apparent to a person skilled in the art that as technology advanced, the basic idea of the invention can be implemented in various ways. The invention and its embodiments are therefore not restricted to the above examples, but they may vary within the scope of the claims.

1.-8. (canceled)

9. An elongated rapid exchange catheter configured to measure fractional flow reserve of a patient, the catheter comprising:
a shaft;
a distal portion coupled to a distal end of the shaft, the distal portion comprising a lumen configured for inserting a guide wire, and an entry via in a side wall of the lumen for inserting the guide wire into the lumen, wherein the entry via is disposed closer to the proximal end of the distal portion than the distal end of the distal portion;
a plurality of sensing devices comprising at least two distal pressure sensing devices disposed at the distal portion on the distal side of the entry via, and a proximal pressure sensing device disposed on the proximal side of the entry via, wherein the at least two distal pressure sensing devices are disposed at mutually different predefined distances from the proximal pressure sensing device along the longitudinal dimension of the catheter,
wherein each of the plurality of sensing devices comprises a pressure sensor and an interface circuitry that performs an analog-to-digital conversion to at least one signal received from the pressure sensor.

10. The rapid exchange catheter according to claim 9, wherein the proximal pressure sensing device is disposed at the distal portion of the catheter between the entry via and the proximal end of the distal portion.

11. The rapid exchange catheter according to claim 9, wherein the proximal pressure sensing device is disposed in a recess within the volume of the shaft at or near the distal end of the shaft.

12. The rapid exchange catheter according to claim 9, wherein the proximal pressure sensing device is disposed in a recess at a junction coupling the shaft and the distal portion.

13. The rapid exchange catheter according to claim 9, wherein any pressure sensing device disposed at the distal portion is disposed in a recess within the volume of the side wall of the distal portion.

14. The catheter according to claim 9, wherein digital signals received from said pressure sensing devices are combined or multiplexed for transmission over an electrical wiring carrying the digital signals to the proximal end of the catheter.

15. The catheter according to claim 9, wherein the at least two distal pressure sensing devices distal are disposed in the distal portion with distance along the longitudinal dimension of the catheter from 1 cm to 5 cm from each other and a distance between the proximal pressure sensing device and any of the at least two distal pressure sensing devices is between 10 and 30 cm.

16. The catheter according to claim 9, wherein the plurality of sensing devices further comprises a flow sensor.
